(19) United States
(12) Patent Application Publication
(10) Pub. No.: US 2013/0144385 A1
(43) Pub. Date: Jun. 6, 2013

(54) PROSTHETIC MENISCI AND METHOD OF IMPLANTING IN THE HUMAN KNEE JOINT

(71) Applicant: Robert Vowles, Romsey (AU)

(72) Inventor: Robert Vowles, Romsey (AU)

(21) Appl. No.: 13/816,489

(22) PCT Filed: Aug. 12, 2011
(86) PCT No.: PCT/AU11/01049

§ 371 Date: Feb. 11, 2013

(30) Foreign Application Priority Data
Aug. 12, 2010 (AU) 2010903553

(51) Int. Cl. A61F 2/08

(52) U.S. Cl. CPC A61F 2/08
USPC 623/14.12

(57) ABSTRACT

Prosthetic knee menisci to be implanted in place of deteriorated native menisci to prevent damage to the articular cartilage of the femoral and tibial condyles and to arrest the progressive development of osteoarthritis; said prosthetic menisci being sized and shaped to be congruent with the femoral and tibial condylar surfaces; being of reinforced construction for strength and durability; being made from materials having elastomeric characteristics similar to those of native menisci; having bearing surfaces treated chemically and/or physically to improve the efficiency of lubrication by synovial fluid; being restricted in translation within the interarticular space by a plurality of locating elements of various forms circumferentially distributed in the zone between said prosthetic menisci and circumferentially-arranged, enclosing locating bands; having optional internal elements for stiffening and anchoring purposes, optional external elements for stiffening purposes and optional straps joining their ends to transmit hoop stresses.

This invention relates generally to surgical methods of alleviating the discomfort and impaired mobility resulting from deterioration or injury to the meniscus of the human knee. More specifically, it relates to a method of replacing either or both of the native menisci in the human knee with prosthetic menisci.

Arthritic deterioration of the knee joint is now extremely common in the western world. A progressively aging population is doubtless a contributory factor. As a result of the limited options available, the number of total knee joint replacements being performed annually in the United States is rapidly becoming an unsustainable burden on the healthcare system. Worldwide, the number of procedures is increasing almost exponentially, despite the fact that it is irreversible and may ultimately require revision. Indeed, the number of total knee replacement revisions has become so great that it now constitutes a well defined sub-speciality in orthopaedic surgery. Clearly, any alternatives to forestall the need for total knee replacement should be considered.

The knee complex is one of the most frequently injured joints in the human body. The knee joint works in conjunction with the hip joint and ankle to support the weight of the body during static, erect posture. Dynamically, it is responsible for moving and supporting the body during a variety of both routine and difficult activities. The fact that the knee must fulfil both major stability and major mobility functions is reflected in its complex structure and functionality.

The two major bones of the leg are the femur, the proximal end of which pivots at the hip joint and the tibia, the distal end of which pivots at the ankle joint. The femur and tibia pivot are joined in an articulated relationship at the knee by the tibiofemoral joint, the largest in the body. The distal end of the femur and proximal end of the tibia are expanded and, although this provides some basis for stability, there is no great adaptation of the bony ends one to another. The distal end of the femur is developed into two discrete condyles, the lower surfaces of which are smoothly rounded and covered in (hyaline) articular cartilage which provides a smooth bearing surface. The anteroposterior convexity of the condyles is not consistently spherical, having a smaller radius of curvature posteriorly. Separated by the intercondylar notch, the condyles have considerable posterior development to accommodate flexing of the knee joint. The medial condyle has greater, posterior development and a greater vertical development which compensates for a degree of obliquity of the shaft of the femur. The lateral femoral condyle is shifted anteriorly in relation to the medial condyle and the articular surface of the lateral condyle is shorter than that of the medial condyle. The proximal end of the tibia comprises shallow, concave lateral and medial plateaus covered with articular cartilage, the medial plateau being larger than the lateral. The tibial plateaus are separated by the lateral and medial intercondylar eminences or tubercles. The femoral condyles are located by and pivotally supported in semi-annular fibro-cartilaginous structures, the menisci, located on the tibial plateaus. These accessory joint structures provide smooth, concave upper surfaces forming complementary bearing surfaces against which the condyles work during articulation of the knee. The knee is also supported by a laterally-located, long auxiliary bone, the fibula. The fibula is strongly bound to the tibia at its distal end, but has a small synovial joint at its upper end joined to the tibial epiphysis. The capsule of the superior tibiofibular joint is reinforced by anterior and posterior ligaments.

The patella (kneecap) is embedded in the quadriceps tendon which connects the quadriceps musculature of the anterior upper thigh to the patella, the patella being connected by the patellar ligament to the tibia just beneath the knee. In simple terms, the posterior surface of the patella is provided with a projection which, during knee flexion, is slidingly displaced in the trochlea, a groove formed in the anterior surface of the femur, between the condyles. The contact zones of patella and femur are covered with smooth articular cartilage providing low friction, complementary working surfaces. The combination of quadriceps tendon, patella and patellar ligament acts rather like a pulley, transmitting forces generated by the quadriceps musculature to the tibia via the flexed knee to straighten the leg or decelerate the rate of flexion. The patella obviously also serves the further function of protecting the knee joint from impact damage.

The knee joint is stabilised by a plurality of ligaments and tendons connecting and/or enclosing its components. During knee joint motion, the fibre bundles of the knee ligaments are non-uniformly loaded in a recruitment pattern which depends on successive relative orientations of the insertion sites. The principal ligaments of the knee are the anterior and posterior cruciate ligaments, the tibial collateral ligament, the fibular collateral ligament, the patellar ligament, the oblique popliteal ligament, and the arcuate popliteal ligament. Of these, the cruciate ligaments are attached, as designated, to the central anterior and posterior surfaces of the tibia and pass obliquely upwards between the condyles to join the femur; acting to substantially locate the condyles on the tibia in the sagittal plane in all knee configurations. The tibial and fibular collateral ligaments are attached, respectively, to the medial and lateral edges of the femur and extend downwardly to join the tibia and fibular capsule; acting to maintain the joint relationship in the coronal plane. The popliteal ligaments provide auxiliary reinforcement of the knee joint. The function of the patellar ligament is explained above. With the knee extended, both the tibial and fibular collateral ligaments, as well as the anterior part of the anterior cruciate ligament, are taut. During extension, the femoral condyles glide into a position which causes complete unfolding of the tibial collateral ligament. During the final 10° of extension, an obligatory terminal rotation is triggered in which the knee is rotated medially 5°. The final rotation is produced by a lateral rotation of the tibia in the non-weight-bearing leg and by a medial rotation of the femur in the weight-bearing leg. This terminal rotation is made possible by the shape of the medial femoral condyle, assisted by the iliotibial tract and is caused by the stretching of the anterior cruciate ligament. Both cruciate ligaments are slightly unwound and both collateral ligaments become taut. In the flexed position, the collateral ligaments are relaxed while the cruciate ligaments are taut. Rotation is controlled by the cruciate ligaments which are brought into twisting contact during medial rotation of the tibia and unwound during its lateral rotation. Because of the oblique configuration and shaping of the crucial ligaments, at least part of one of them is always in tension, controlling the joint during relaxation of the collateral ligaments. The tibial collateral ligament also acts to limit medial rotation. While the heads of the gastrocnemius muscles, the muscles of the posterior calf, pass behind the knee, most muscles above and below the knee joint exercise their functions via aponeuroses—thin sheets of tendinous material which substantially surround the knee—which are thickened locally where higher forces are transmitted. A high degree of blending and inter-attachment occurs between tendons and ligaments, and nerves and blood vessels extend throughout the various tissues of the knee area. The fact that almost all tendons and other tissue surrounding the knee lie parallel to the bones and move lengthwise across the joint creates a potential for substantial frictional forces. This explains the existence of numerous bursae in the knee area—essentially thin-walled capsules filled with synovial fluid which act to reduce friction between independently moving tissue structures. Some bursae communicate with or are contiguous with the synovial membrane.

The articular cavity of the knee joint is the largest joint space of the body and is completely enclosed by the joint capsule. The capsule is reinforced medially, laterally and posteriorly by capsular ligaments and is critical in restricting excessive joint motions to maintain joint integrity and normal function. In general, the outer or fibrous portion of the capsule is firmly attached to the inferior aspect of the femur and the superior portion of the tibia. Posteriorly, the capsule is attached proximally to the posterior margins of the femoral condyles and intercondylar notch and distally to the posterior tibial condyle. The patella and its associated structures complete the anterior portion of the capsule. The capsule is strongly innervated with mechanoreceptors which may contribute to muscular stabilisation of the knee joint by initiating reflex-mediated muscular responses. The synovial membrane forms an inner lining of much of the knee joint capsule. Its purpose is to secrete synovial fluid into the joint space for the lubrication and nutrition of avascular structures, such as the menisci. The membrane is complex in its arrangement, passing beneath the patella and, posteriorly, breaking away from the capsule and invaginating anteriorly to exclude the cruciate ligaments. The tribological interrelationship between the synovial fluid and the cartilage surfaces which it lubricates is a complex one and a unified model of joint lubrication is yet to be proposed.

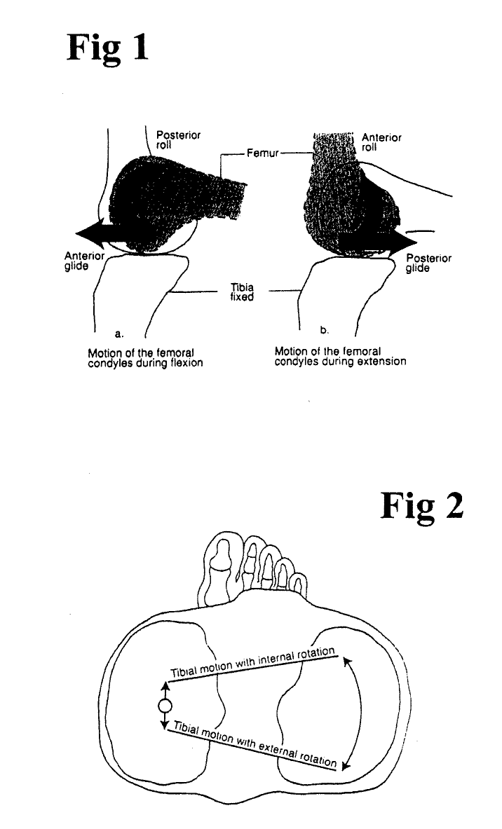

During displacement of the tibiofemoral joint, rotatory or angular motion occurs about changeable but definable axes. In addition to the angular motion, translation in an anteroposterior direction is common on both the medial and lateral tibial plateaus. To a lesser extent, medial and lateral translations can occur in response to varus (tending to a knock-kneed posture) and valgus (tending to a bow-legged posture) forces. The small amounts of anteroposterior and medial/lateral displacements that occur in the normal knee are the result of joint incongruence and variations in ligamentous elasticity. Although these translations may be seen as undesirable, they are necessary for normal joint motion to occur. The axis for tibiofemoral flexion and extension can be simplified as a line passing more or less horizontally through the approximate centres of curvature of the articular surfaces of the femoral condyles. However, this axis is not fixed and shifts throughout the range of motion, principally as a result of incongruence of the joint surfaces. The large articular surface of the femur and the relatively small tibial condyle create a potential problem as the femur commences rotation on the tibia. During extended flexion, in order for them to remain on the tibial plateaus, the femoral condyles must simultaneously glide anteriorly and, during extension, simultaneously glide posteriorly. As stated, the cruciate ligaments act to substantially locate the condyles on the tibia during flexion and extension. As illustrated in a. of FIG. 1, during flexion of the knee joint, tension applied by the anterior cruciate ligament restrains the condyles from posterior displacement. Similarly, as illustrated in b. of FIG. 1, during extension of the knee joint, the posterior cruciate ligament restrains the condyles from anterior displacement. These effects are reinforced by the capsule and the layers of ligamentous and tendinous tissue surrounding the knee joint. For example, the iliotibial band, which transmits forces from thigh muscles to the tibia, provides lateral support to the knee joint and, during flexion, restricts excessive anterior translation of the tibia under the femur.

Medial and lateral rotation of the knee joint are angular motions that are named for the motion (or relative motion) of the tibia on the femur. These axial rotations of the knee joint occur about a longitudinal axis that runs through or close to the medial tibial intercondylar tubercle. Consequently, the medial condyles acts as pivot points while the lateral condyles move through a greater arc of motion, regardless of the direction of rotation. This is illustrated in FIG. 2. As the tibia laterally rotates on the femur, the medial tibial condyle moves only slightly anteriorly on the relatively fixed medial femoral condyle, whereas the lateral tibial condyle moves a larger distance posteriorly on the relatively fixed lateral femoral condyle. During tibial medial rotation, the medial tibial condyle moves only slightly posteriorly, whereas the lateral condyle moves anteriorly through a longer arc of motion. During both medial and lateral rotation, the knee joint menisci will distort in the direction of movement of the corresponding femoral condyle and, therefore, maintain their relationship to the femoral condyles as they did in flexion and extension. The range of knee joint rotation possible depends upon the flexion/extension position of the knee. When the knee is in full extension, the ligaments are taut, the tibial tubercles are lodged in the intercondylar notch and the menisci are tightly interposed between the articulating surfaces; consequently, little axial rotation is possible. As the knee flexes towards 90 degrees, capsular and ligamentous laxity increase, the tibial tubercles are no longer in the intercondylar notch, and the condyles of the tibia and femur are free to move in relation to each other. The maximum range of axial rotation is available at 90 degrees of knee flexion: the range of lateral rotation being 0 to 20 degrees and the range of medial rotation, 0 to 15 degrees, giving a total medial/lateral rotation of up to 35 degrees.

The knee menisci were once thought to be just a form of vestigial tissue but are now understood to be vital to the proper functioning of the knee joint. In addition to enhancing joint congruence, the menisci play an important role in distributing forces through the knee, in reducing friction between the femur and tibia and in absorbing shock loadings to the knee. The menisci cover between one half and two thirds of the tibial articular plateau and are open towards the tibial tubercles, the lateral meniscus covering a greater percentage of the smaller lateral tibial plateau. As a result of its larger exposed surface, the medial condyle has a greater susceptibility to the enormous compressive loads that pass through it during routine activities. Although compressive forces in the knee may reach one or two times body weight during gait and stair climbing and three to four time body weight during running, the menisci assume 50% to 70% of this imposed load. Meniscal motion on the tibia is limited by multiple attachments to surrounding structures, some common to both menisci and some unique to each. To accommodate deviations of the femoral condyles from sphericality, the menisci obviously possess some freedom of movement. The medial meniscus has greater ligamentous and capsular constraints, limiting its translation to a greater extent than that of the lateral meniscus. The relative lack of mobility of the medial meniscus may contribute to its greater incidence of injury—some nine times greater than that of the lateral meniscus.

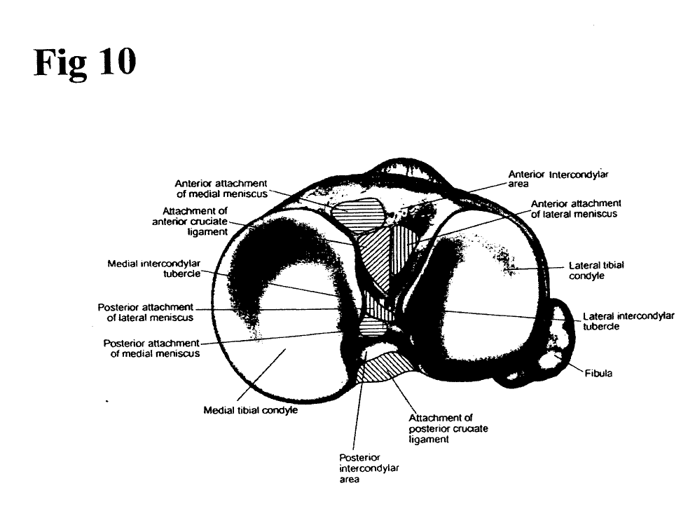

The menisci are best described as crescent-shaped wedges of fibrocartilage supported upon the peripheral aspects of the articular surfaces of the proximal tibia. They function to effectively deepen the medial and lateral tibial fossae for articulation with the condyles of the femur. They are thickest at their external margins and taper to thin, unattached edges as they extend radially inwards. The superior surfaces of the menisci are slightly concave to accommodate the condyles of the femur and providing greater contact surface area. The medial meniscus is larger than the lateral and more ovoid in shape. Anteriorly, it is thin and pointed at its attachment in the anterior intercondylar area of the tibia, directly outside the anterior cruciate ligament. Posteriorly, it is broadest, attaching in the corresponding posterior fossa, anteriorly to the origin of the posterior cruciate ligament. The lateral meniscus is smaller and more circular, its anterior horn being attached in the anterior intercondylar area, posteriorly and laterally to the insertion of the anterior cruciate ligament. Its posterior horn terminates in the posterior intercondylar area, immediately anterior to the termination of the posterior horn of the medial meniscus. The lateral meniscus is weakly attached around the margin of the lateral tibial condyle, except where crossed by the popliteal tendon and is not attached to the fibular collateral ligament. Near its posterior attachment, the lateral meniscus frequently sends off a collection of fibres which either join or lie behind the posterior cruciate ligament. The bundle of fibres, termed the posterior meniscofemoral ligament, terminates in the medial condyle of the femur immediately behind the attachment of the posterior cruciate ligament. Depending upon whether it passes anteriorly or posteriorly to the posterior cruciate ligament, the ligament is known, respectively, as the ligament of Humphry or the ligament of Wrisberg. Occasionally, both meniscofemoral ligaments are present, their function apparently being to provide a secondary restraint to posterior tibial translation. Occasionally, an anterior meniscofemoral ligament is also present, with a similar but anterior relationship to the posterior cruciate ligament. The lateral meniscus is thus loosely attached to the tibia and has frequent attachment to the femur. Therefore, it tends to move forward and backward with the lateral femoral condyle during flexion of the knee. In contrast, the medial meniscus is more firmly fixed to the tibia. The convex anterior margin of the lateral meniscus is connected to the anterior horn of the medial meniscus (or its convex anterior margin) by the transverse genicular ligament. This connection allows the two menisci to move in unison. This ligament, which varies considerably in thickness, is often absent. The curved external margins of the menisci are attached to the fibrous capsule of the knee joint (and thus the synovial membrane) and through it, to the edges of the articular surfaces of the tibia. The capsular fibres attaching the meniscal margins to the tibial condyles are termed coronary ligaments. The medial meniscus is further restrained by its attachment to the deep surface of the tibial collateral ligament. The capsular and tibial attachments of the meniscus may be seen clearly in FIG. 9. The tibial plateaux and meniscal horn attachment sites are illustrated in FIG. 10.

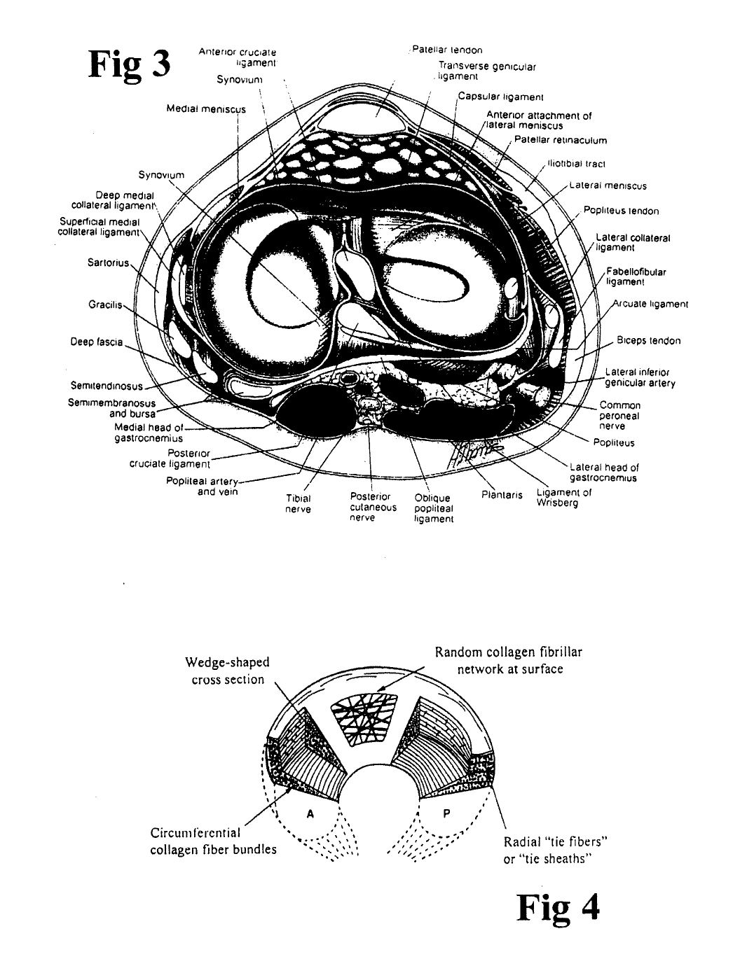

The thick peripheral margins of the menisci have an extensive microvascular network that arises from their respective superior and inferior genicular branches of the popliteal artery, while the thin, unattached edges of the menisci within the joint are avascular. The perimeniscal capillary plexus is oriented circumferentially and it branches extensively into smaller vessels to supply the menisci. The capillaries are developed into smaller vessels which extend peripherally throughout 10 to 30 percent of the medial meniscus and 10 to 25 percent of the lateral meniscus. Similarly, nerve fibres originate in the perimeniscal tissues and radiate into the peripheral 30 percent of the menisci. The most densely innervated regions are the anterior and posterior horns, these nerves being thought to play a proprioceptive role for protective neuromuscular reflex control of joint motion and loading. The location and morphology of the menisci and associated structures are illustrated in FIG. 3.

The anterior glide of the femoral condyles during flexion is also influenced by the menisci. The effective ‘wedging’ effect of the menisci acts to restrain the condyles from posterior displacement while the reaction forces applied to them act to displace the menisci posteriorly on the tibial plateaus. Deformation of the menisci occurs because the rigid attachment of their horns limits their ability to move in their entirety. Posterior deformation permits the menisci to remain beneath the femoral condyles as the condyles move on the tibial plateaus. As the knee returns to extension from full flexion, the menisci return to their neutral positions and, as extension continues, are deformed anteriorly. Appropriate posterior deformation of the menisci is assisted by muscular mechanisms. During knee flexion, for example, through its attachment to posterior horn of the medial meniscus, the semimembranous applies a force to the medial meniscus urging it posteriorly. An investigation has found that, in more than 40 percent of knees, the semimembranous has a similar attachment to the posterior horn of the lateral meniscus. The popliteus applies a similar force to the lateral meniscus.

The menisci are, effectively, cartilaginous extensions of the tibia composed principally of type I collagen. Water accounts for more than 70 percent of the total weight of the meniscus. Collagen makes up the largest organic content in cartilaginous tissue—some 10 to 20 percent of the wet weight of the extracellular matrix. Currently, more than 20 types of collagen have been identified, based upon their specific amino acid sequences. The basic molecular structure of collagen begins with three intertwined alpha helical polypeptide chains bound together through covalent cross-links. These tropocollagen molecules, as they are termed, then self-aggregate into a quarter-stagger manner to form fibrils with a characteristic 64 to 100 nm banding visible under electromicroscopy. These collagen fibres further aggregate into small-diameter fibrils 10 to 25 nm in width and larger-diameter fibres 1 to 2 μm in width, depending upon the collagen type and location. Proteoglycan aggrecans constitute the second largest part of the organic material of cartilaginous tissues, accounting for some 1 to 2 percent of the weight of the meniscus. An aggrecan consisting of a long protein core to which approximately 150 glycosaminglycan (GAG) chains are attached. Sulphated GAGs found in cartilage are chondroitin sulphate, keratan sulphate and hyaluronic acid (HA). Components of proteoglycan are produced separately by the chondrocytes and extruded into the pericellular matrix in a form soluble in the interstitial fluid. Subsequent aggregates become more securely immobilised in the interfibrillar space of the surrounding collagen network and are held in place principally by frictional interactions. The result is a strong, cohesive, porous-permeable, fibre-reinforced composite material.

With reference to FIG. 4, the physical structure of the meniscal collagen networks can be roughly divided into three separate zones. In the outer, superficial layer, fibrils are randomly oriented and are interwoven to form a fine mesh. Immediately beneath this mesh is a narrow zone in which the collagen bundles show a much more irregular orientation. Interior to these two surface zones, the collagen fibres form large bundles that can be seen with the naked eye. These fibre bundles are circumferentially arranged, extending from the anterior attachment site to the posterior attachment site. Between these large, circumferentially arranged collagen fibre bundles are smaller tie fibres or tie sheaths orientated radially and extending from the periphery to the inner edge. Thus, compressive force applied to the meniscus is translated into a circumferentially directed tensile or hoop stress, supported by the strong circumferential fibres that dominate its ultrastructure. Viscoelastic behaviour of meniscus material to tensile and compressive forces is complex, the tensile modulus, stiffness and failure stress correlating with collagen content and ratio of collagen to proteoglycan (PG). When meniscus material is loaded in compression, a loss of volume can occur due to fluid exudation from the tissue and/or fluid distribution within the tissue. The concentration of PG within the tissue has been shown to affect permeability, suggesting a direct relationship between PG content and compressive stiffness. The concentration and molecular conformation of proteoglycan aggregates in cartilage vary with age and disease and the amount of PG present depends on joint loading and motion. In general, with aging and disease, the size of the PG aggregates decreases by shortening of the hyaluronic acid chain or by shortening of either the protein core or glycosaminglycan chains, or both. Another important age-related change in PG can be observed at the molecular level. Chondroitin sulphate (CS) has two isomeric forms, CS₄ and CS₆, where the subscript indicates the location of sulphation on the hexosamine. It has been observed that the CS₄ isomer is more common in young cartilage, whereas the presence of CS₆ isomer increases with age. The net result is a reduction in resilience of the cartilage and a concomitant disposition towards mechanical damage.

The lubrication process of the knee joint is thought to be a combination of boundary lubrication and fluid film lubrication, but modified by the characteristics of the articulating cartilage surfaces. Boundary lubrication depends upon the chemical adsorption of a monolayer of lubricant molecules to the articulating surfaces, the clearance between the articulating surfaces maintained by the lubrication mechanism being as small as only a few μm. During relative motion, the surfaces are protected by the lubricant molecules sliding over one another, preventing adhesion and abrasion of the naturally occurring surface asperities. In fluid film lubrication, a much thicker (10-20 μm) layer of lubricant is necessary, compared with the molecular size of the lubricating glycoprotein molecule. The lubricant layer causes relatively wide separation of the articulating surfaces compared with the typical surface roughness of normal articular cartilage. The load applied across the surfaces is supported by pressure generated in the fluid film. The low relative speed difference of the articulating surfaces and the high loads applied across them are, generally speaking, incompatible with the concept of fluid film lubrication. This has led to the postulation of a ‘weeping lubrication’ process in which lubricant exudes from the permeable cartilage surface as a result of applied pressure. Experimental investigations have been unable to validate this hypothesis and a theory of ‘boosted lubrication’ is now accepted. In this process, high pressures generated in the fluid lubricant film causes synovial fluid without hyaluronate to flow into the cartilage tissue, leaving a concentrated gel in the gap to protect the articulating surfaces. It is also now accepted that micro-elastohydrodynamic lubrication contributes substantially to formation of effective lubricating films in synovial joints. Micro-elastohydrodynamic lubrication occurs when elastomeric layers deform under pressure, forming a fluid film in which asperities in the articulating surfaces are flattened as a result of local pressure perturbations. In light of the ability of synovial joints to maintain a high level of lubrication efficiency under a wide range of conditions, it is not unreasonable to infer the presence of a hybrid lubrication mechanism and it is notable that substantial differences of agreement still exist in relation to the subject. Regardless of the lubricating mechanism, by engineering standards, friction in the diarthrodial joint is reduced to levels associated with a fluid film separating the sliding surfaces (hydrodynamic lubrication), but at sliding velocities normally associated only with boundary (solid-to-solid) lubrication and, hence, with frictional levels one to two orders of magnitude higher. As an indicator of the efficiency of the lubrication system, coefficients of kinetic friction (g) in human joints are approximately 0.002 to 0.006, compared with a value of 0.04 for Teflon, which is one of the best boundary lubricants used in non-biological systems. The coefficient of friction (μ) is the ratio of the frictional force (T) resisting movement of one articulating surface over another and the normal force (N) urging the articulating surfaces together (μ=T/N).

The search for a boundary lubricant with the capability to reduce friction to the remarkably low levels mentioned in the preceding paragraph has attracted much attention. Hyaluronic acid has long been recognised for its remarkable ability to retain water and control the viscosity of synovial fluid, but its failure to lubricate under any appreciable load has ruled it out as the key. Experimental centrifugation of synovial fluid demonstrated that the active load-bearing constituent was located in the ‘proteinacious’ layer, rather than the ‘hyaluronate’ layer. While further research identified within the load-bearing fraction a glycoprotein unique to synovial fluid, calculations of the molecular weight of lubricin', as it was termed, failed to account for some 9 to 13 percent. The view was adopted that a component of lubricin is deposited onto the articular surface from the synovial fluid. This adsorption theory is reinforced by the fact that lubrication of a surface exposed to synovial fluid is not immediately compromised when synovial fluid is replaced with saline. Moreover, a surface must be in contact with synovial fluid for approximately three minutes before it is fully lubricated. The identification of surface-active phospholipid (SAPL) in association with other sliding surfaces in the body, namely the pleura and pericardium, led researchers to seek similar compounds in the joints. At the molecular level, the predominant surface-active component was identified as the surfactant, L-d-dipalmitoylphosphatidylcholine (DPPC). Subsequent studies have shown DPPC and synovial SAPL to be capable of reducing friction to the very low levels (μ=0.001-0.006) characteristic of the mammalian joint and of doing so at low sliding velocities and under high load. While the production of surfactant by soft tissues surrounding the joint has been demonstrated, direct adsorption of SAPL from the synovial fluid should be severely restricted by its very low solubility in water and, hence, in synovial fluid. This difficulty would be overcome if a highly soluble macromolecule were to be present as a carrier. It is speculated that lubricin might provide that carrier.

Examination of the knee joint using magnetic resonance imaging has shown the relatively large excursions experienced by the menisci during various phases of knee joint flexion. FIGS. 6, 7 and 8 give some figures in this regard. Given the relatively high degree of meniscal mobility, it will be appreciated that the primary locational mechanism of the femoral condyles on the tibial plateaux is tension applied by the anterior and posterior cruciate ligaments. The menisci essentially provide a moveable, cushioned bearing surface for the femoral condyles and may, at extremes of knee flexion, provide supplementary locational assistance. This factor makes possible the provision of prosthetic menisci which, while not able to accommodate the rigors of athletic performance, will readily meet the needs of a sedentary person of middle age.

Meniscus failure commonly takes two forms: direct mechanical damage and that resulting from degenerative breakdown. In sports persons, for example, acute tearing of the meniscus may result when the knee is bent and forcefully twisted. Degenerative tears in the meniscus are very common in older persons with some 60 percent of western populations over the age of 65 years having some sort of degenerative breakdown. While acute tearing may result in the sudden onset of symptoms, in older subjects, degenerative breakdown may result from minor events and be symptomless for an extended period. A combination of circumstances, such as age-related degenerative changes, ‘wear and tear’ arthritis of the whole knee typically found in former athletes, inflammatory arthritis, decline of synovial lubrication, degradation caused by enzymes, unnatural gait, alignment disorders of the leg or excessive knee loadings as a result of occupational activities may result in progressive frictional wear of a meniscus, the cartilage having very little power of natural restoration. The meniscus is capable of self healing only in the vascularised, innervated peripheral zone while the unattached central zone is nourished only by synovial fluid and, generally speaking, is incapable of self healing.

Interventions to alleviate the effects of cartilage injury or failure take a number of forms and include:
Flexibility exercise programmes, ice packs, unloading braces
Analgesics, anti-inflammatory drugs, intra-articular injection, needle lavage or acupuncture
Viscosupplementation
Arthroscopy, meniscectomy
Osteotomy
Meniscus replacement—allograft
Meniscus replacement—growth in-vivo
Meniscus replacement—growth in-vitro
Meniscus replacement—prosthetic
Knee joint replacement—unicapsular
Knee joint replacement—total
   Those in the first group are self-explanatory. Those in the second group are self-explanatory, excepting needle lavage. This procedure involves washing out the knee joint with a sterile saline solution, typically followed by an injection of a corticosteroid into the joint. The effect of the procedure is variable. In viscosupplementation, a preparation of hyaluronic acid is injected into the knee joint. This restores the depleted lubrication commonly found in subjects with osteoarthritis and has been found to relieve pain.

Arthroscopy, performed with an arthroscope (a type of endoscope) inserted into the joint through a small incision, can be performed to evaluate and treat a range of orthopaedic conditions. Arthroscopic treatment may include repair or partial removal of the meniscus, anterior cruciate ligament reconstruction or articular cartilage repair. Where a meniscus is damaged beyond repair or partial removal, a total meniscectomy may be performed. This option is avoided wherever possible, owing to the increased risk of osteoarthritis leading, ultimately, to the need for total knee joint replacement. Meniscal repairs are normally limited to the young and to damage in the vascularised zone and are effected by suturing or the use of small fixation darts, pins or clips of a bio-absorbable material. Some success has been achieved with meniscal repairs in the avascular zone with the use of exogenous fibrin clots. Depending upon the type of treatment received, recovery of full use of the knee may be rapid or slow. In the case of a meniscus repair, use of a knee brace may be specified. Patients can normally bear weight on the affected knee a day or two after the surgery and return to full activity within two to four weeks. A return to vigorous sporting activity may be delayed for several months.

Osteotomy has been employed successfully in younger patients who have sustained osteoarthritis on one side of the knee. The procedure involves the removal of a wedge-shaped section of bone from the appropriate side of the tibia immediately beneath the knee joint. This permits correction of a mal-alignment of the knee joint causing the arthritic condition, reducing the load on the deteriorated compartment and, frequently, stimulating the blood flow to it. The adjusted position of the tibial plateau is stabilised with a plate. Rehabilitation may involve the use of a continuous passive motion machine immediately after surgery to reduce stiffness, ease pain, prevent blood clots from forming and prevent extra scar tissue from forming inside the joint. Hospital stay may be several days, a patient usually being discharged when able to safely get in and out of bed and walk with crutches or a walking frame. Exercises will be prescribed to ensure the regaining of good contraction of the quadriceps muscle and an improved range of knee motion. It is common for patients to wear a knee brace for up to six weeks following surgery to protect the knee joint. Stitches are commonly removed in 10 to 14 days with full recovery in two to three months. In the best of circumstances, a tibial osteotomy is considered only temporary, the benefits of the operation usually lasting for five to seven years before a total knee replacement becomes necessary. The procedure may not provide complete pain relief and there are a number of possible complications, fortunately quite rare.

Also in the younger patient, where meniscal preservation is not possible, the implantation of an allograft has achieved some success. An allograft is a cadaveric meniscus which has been selected for size and sterilised by gamma radiation. Following removal of the defective meniscus, the allograft is implanted by securing the horns to a bone bridge or plugs inserted into the tibia to provide correct location and by suturing its outer edge to the capsule or edge of the tibial plateau. Patients are normally discharged on the day of the procedure and analgesics and anti-inflammatory drugs may be required for four to seven days. A cryocuff is commonly employed to reduce swelling. Patients are encouraged to do straight leg raises in the brace immediately after surgery. A brace is used to walk with the knee in extension for six weeks. Range of motion is generally started soon after surgery from 0-90 degrees, without any weight-bearing during motion. The brace is unlocked at six weeks and weaned off after eight weeks when good quadriceps control is demonstrated. Motion is increased as tolerated at six weeks, but deep squats are avoided until 12 weeks. Low impact type activities such as swimming and exercise machines are encouraged at 12 weeks, with advancement to cutting and pivoting sports generally at 16 weeks. The assistance of a physical therapist is very helpful in achieving a rapid full recovery. As with osteotomy, allograft transplantation has a number of possible complications and is not always successful.

Considerable experimental effort has been directed towards the in-vivo growth of meniscal material. This tissue engineering technology involves the use of biological or synthetic matrices. The process aims at growing on the matrix chondrocytes recruited from the remaining meniscus or seeded into the matrix before its implantation into the joint. A C-shaped disk of suitable matrix material is created, the damaged meniscal tissue is debrided until healthy, vascularised tissue is exposed and the implant is trimmed to shape and sutured into place. The matrix implant is intended to be absorbed over time. Although the technology is still at an experimental stage, the use of collagen meniscal implants has achieved some success and has been approved for use in a number of countries. Generally speaking, the resultant cartilage lacks the microstructure and biomechanical characteristics of native cartilage. It is doubtful that the technology can be employed to replace a complete meniscus, due to difficulty in creating fixation methods for re-grown menisci. It is also doubtful that the regenerated cartilage material would have the requisite strength and durability. Recovery and rehabilitation following this procedure are unlikely to be less onerous than that following an allograft implantation.

Similarly, considerable experimental effort has been directed towards the in-vitro growth of meniscal material. The tissue engineering technology involved is similar in nature to that of the in-vivo technique, excepting that the material is moulded and regularly subjected to aggressive tension and compression with a view to encouraging the development of a microstructure similar in nature to that of the native meniscus. The implantation procedure is similar to that used to implant matrix material for the in-vivo generation of cartilage. This technology is also still at an experimental stage, but shows considerable promise. Assuming the eventual implantation of in-vitro-created menisci, recovery and rehabilitation following the procedure are also unlikely to be less onerous than that following an allograft implantation. It is estimated that patients will be required to avoid weight-bearing activities on the affected knee for up to six weeks and may require the use of passive continuous motion during this period.

Considerable experimental work has also been conducted into autograft meniscus replacement. The most common method involves the harvesting of tendinous material (normally the free middle third of the patellar tendon), shaping it and implanting it in a manner similar to that used with matrix material for the in-vivo generation of cartilage. Examination of menisci formed in this way in animal studies have shown good shaping but that the cartilage does not have the microstructure and strength of the native meniscus. This technology is also still at an experimental stage, but must be regarded as promising. Should the procedure become practical for human use, recovery and rehabilitation following the procedure are also unlikely to be less onerous than that following an allograft implantation. Patients are normally required to avoid exposing the affected knee to any weight-bearing activity for at least one month.

Suggestions have been made for bio-compatible polymers and polypeptide materials to be injected into an arthritic joint where they would set and create a supporting surface similar in character to the native meniscus. These methods are also still at an experimental stage, but are unlikely to be effective for other than the restoration of small areas of lost cartilage. Proposals have been made for the implantation of prosthetic menisci, but difficulties have been experienced with locating these and creating a material of sufficient strength and durability. Some of the procedures for replacement of menisci may be performed arthroscopically.

Where one or both knee compartments have suffered irretrievable arthritic deterioration, it is common for a unicapsular or total knee replacement to be pertbrmed. In a typical form of this procedure, the joint is opened and the appropriate femoral and tibial condyles are cut away. Elements of a mechanical joint are fixed to the distal end of the femur and the proximal end of the tibia. Both elements are normally made from a suitable metal alloy material and the femoral unit is provided at its distal end with one or more curved surfaces homologous with the femoral condyles. The tibial unit replicates the tibial plateaux and incorporates a bearing surface homologous with the menisci and tibial articular cartilage. The bearing unit normally takes the form of a plate of high molecular weight polyethylene. The surgical procedure is significant. It involves a complete opening of the knee joint and may extend to several hours. A blood transfusion may be required. Physical therapy is an important part of the recovery process and normally commences 48 hours after surgery. The use of a continuous passive motion is commonly prescribed and some degree of pain, discomfort and stiffness is to be expected during therapy. Patients are normally discharged from hospital on the third or fourth day. Therapy in various forms will continue for several months to minimise scarring and ensure full joint movement. The time to full recovery varies from patient to patent, but may require up to 12 weeks. Risks associated with the procedure are not insignificant and include deep venous thrombosis, pain, post-surgical infection, stiffness, unequal limb length and loosening of the prostheses. An important consequence of knee joint replacement is the fact that, at any time, a prosthesis may become the focus of an invasive infection. As a routine precaution, persons with artificial joints are recommended to take antibiotics before any invasive procedure, including dental.

In light of the foregoing generally, it is postulated that, if arthritic deterioration can be detected at an early enough stage, intervention in the form of the implantation of prosthetic menisci may be sufficient to almost immediately restore normal knee function. Further, this has the potential to arrest the deterioration process and to forestall the eventual need for total or partial knee replacement. If the implantation procedure can be performed arthroscopically as a day procedure, the reduction in demand for hospital bed space and orthopaedic, anaesthetic, physiotherapy and general medical services will represent a significant cost saving. Concomitant benefits would be rapid patient recovery with minimal discomfort and, if required, ease of revision.

Primary objects of the present invention are to provide prosthetic menisci to replace the native human knee menisci, together with surgical procedures for removal of the native menisci and implantation of the prostheses; the prostheses being readily matchable to the dimensions of the femoral condyles, able to be securely located on the tibial plateaus and to replicate normal meniscal motion while providing durable working surfaces of low friction; having compatibility with the constituents of synovial fluid, and capable of accommodating the stresses imposed by the knee working under all normal loads; the surgical implantation procedures and effect of the prosthetic menisci being such as to require minimal rehabilitation for each patient. A secondary object of the present invention is the provision of a prosthetic meniscus which may be implanted using arthroscopic surgical procedures. A tertiary object of the present invention is the provision of a soft and protective prosthetic meniscus which may be temporarily implanted during repair of the femoral or tibial articular cartilage.

According to the present invention, prosthetic menisci of correct size and shape are made from suitable materials and treated to render their surfaces attractive to the lubricating constituents of synovial fluid. Access is gained to the knee compartment via minimal incisions and displacement of the tendinous and capsular tissue surrounding the joint. The native menisci are removed by surgically severing all of their tibial and capsular attachments with careful attention to haemostasis. The prosthetic menisci are correctly positioned between the femoral condyles and the tibial plateaux and secured in place by several means. The synovial capsule is then modified as required to fully enclose the joint, separated tissue is reinstated and the skin incisions are closed.

The various aspects of the present invention will be more readily understood by reference to the following description of preferred embodiments given in relation to the accompanying drawings in which:

FIGS. 1(a) and 1(b) are lateral, diagrammatic views of the bones of the knee during extension and flexion;

FIG. 2 is a superior, diagrammatic view of the proximal end of the tibia of the right knee;

FIG. 3 is a superior, diagrammatic, transverse cross-sectional view through the right knee just above the menisci;

FIG. 4 is a fragmentary, diagrammatic view of a meniscus partially sectioned to display its internal structure;

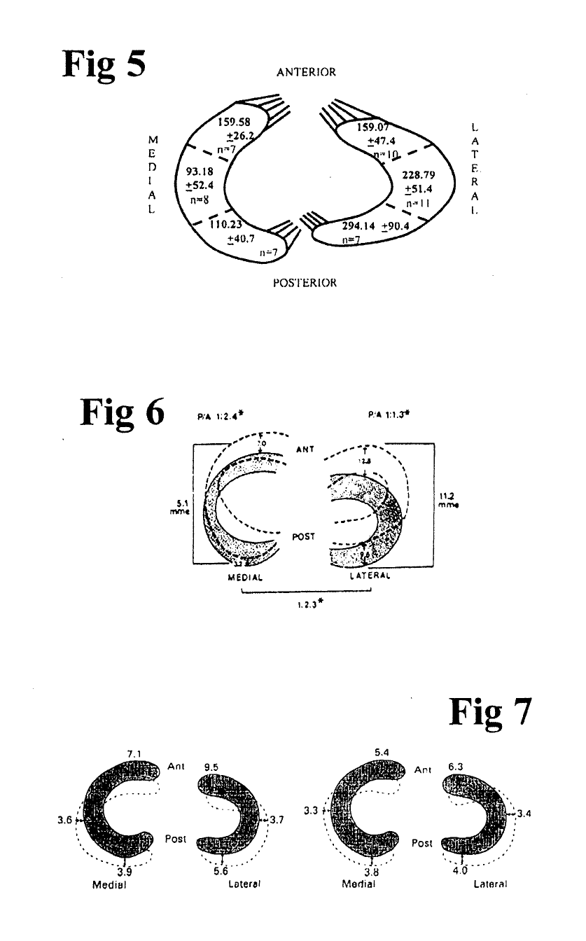

FIG. 5 is a diagrammatic presentation of regional variations of Young's Modulus in the human meniscus in tension;

FIG. 6 is a superior, diagrammatic view of displacement of menisci on the tibial plateaux at 0 and 120 degrees of knee flexion, the displaced positions depicted in solid line¹;

FIG. 7 is a superior, diagrammatic view of displacement of menisci on the tibial plateaux during 90 degrees of flexion from an erect stance with weight bearing and during 90 degrees of flexion in a sitting position, relaxed and bearing no weight, the displaced positions depicted in broken line²;

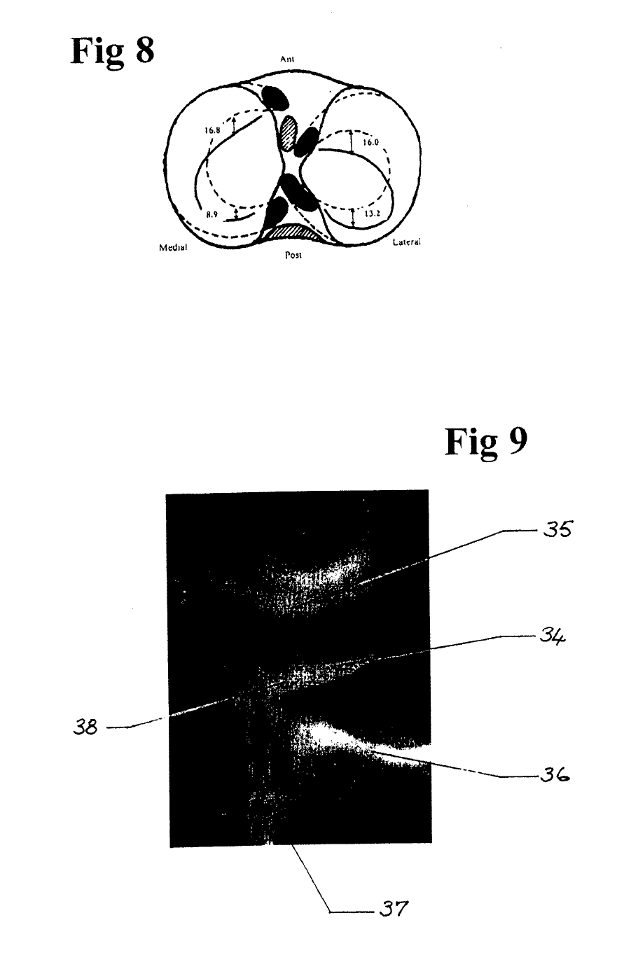

FIG. 8 is a superior, diagrammatic view of displacement of the meniscal inner margins on the tibial plateaux during deep knee flexion, the displaced margins depicted in solid line³;

FIG. 9 is a fragmentary cross-sectional view on a sagittal plane of the femoral condyle, meniscus and tibial plateau, separated to allow an appreciation of their relative dimensions;

FIG. 10 is a superior, diagrammatic view of the proximal end of the tibia of the right knee;

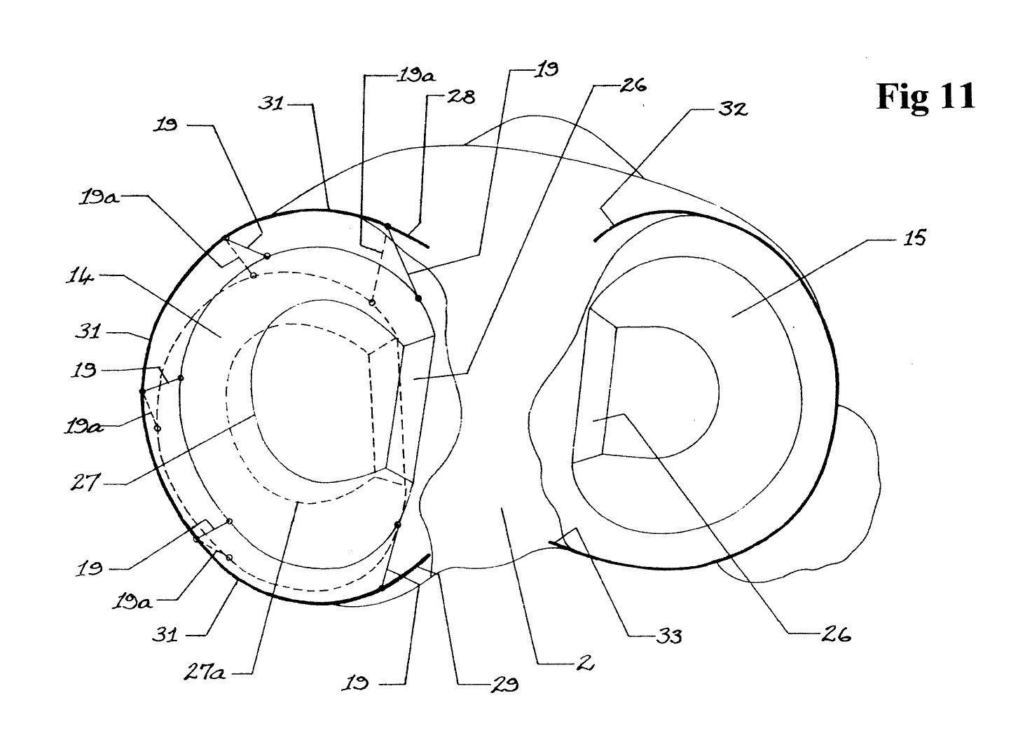

FIG. 11 is the view of FIG. 10 showing the location of supporting frames and positions of prosthetic menisci supported by them;

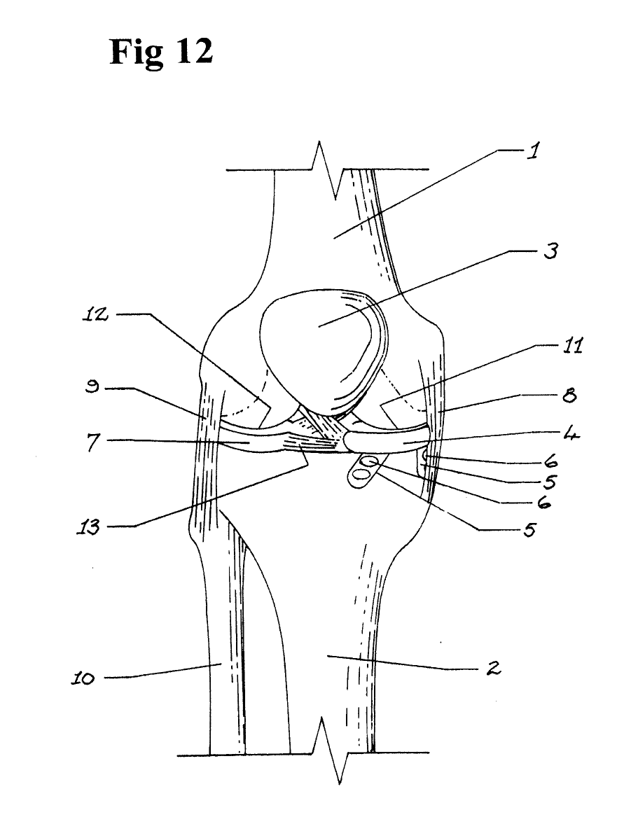

FIG. 12 is an anterior view of the bones of the knee joint depicting a native meniscus and a prosthetic meniscal installation;

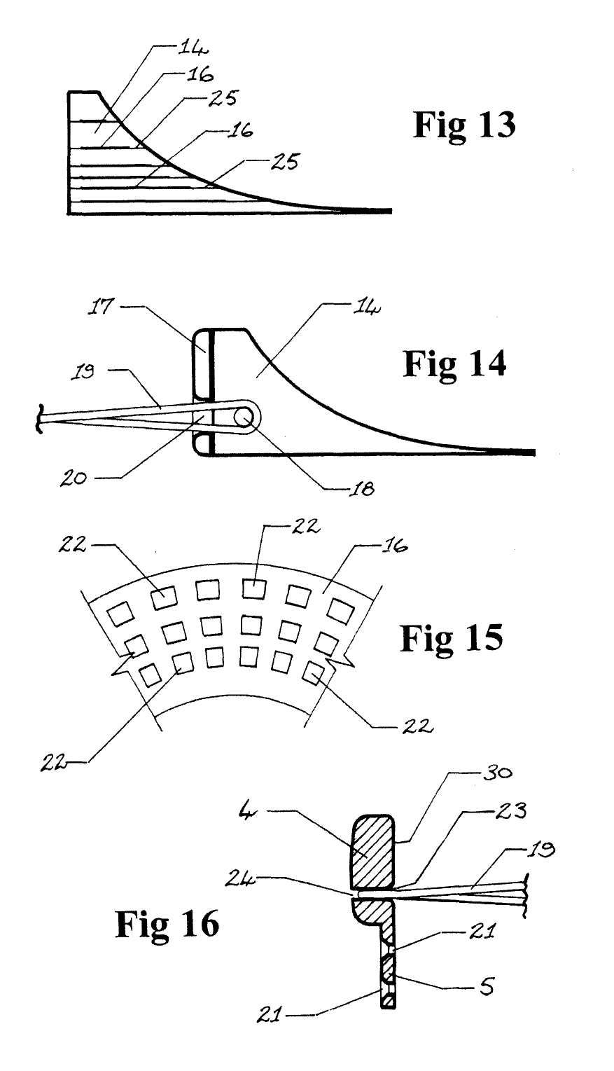

FIG. 13 is a transverse cross-sectional view of a representative prosthetic meniscus, showing laminated construction;

FIG. 14 is a transverse cross-sectional view of a representative prosthetic meniscus, showing an edge stiffening band, stiffening wire and tethers;

FIG. 15 is a fragmentary view on face of typical sheet reinforcing material for prosthetic menisci;

FIG. 16 is a transverse cross-sectional view of a meniscus locating band through its attachment lug;

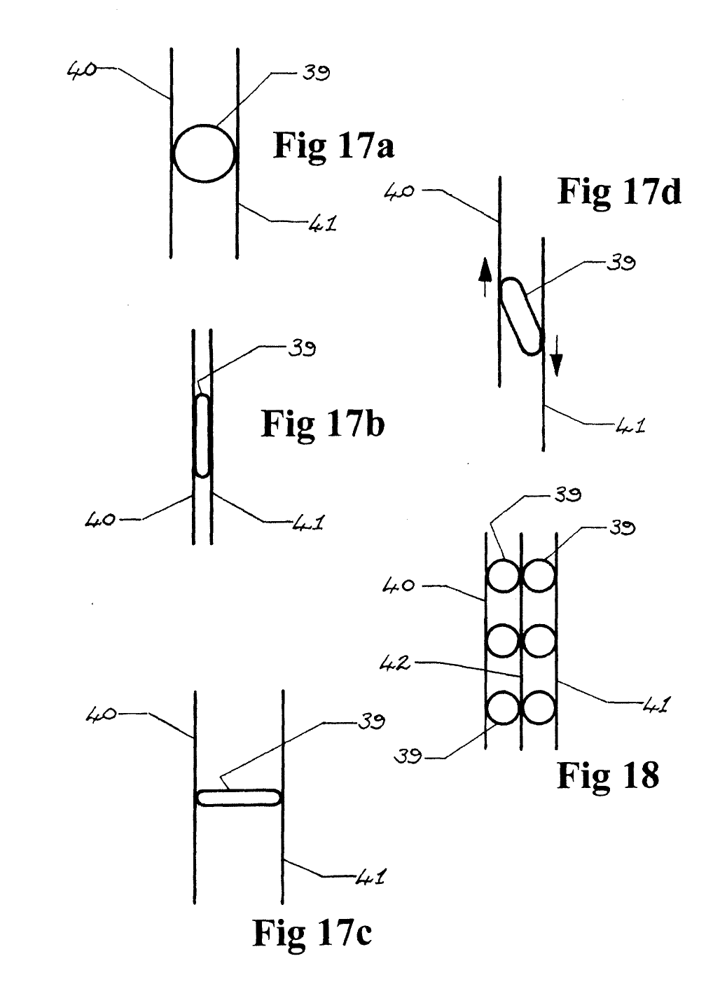

FIGS. 17a, 17b, 17c and 17d depict, in fragmentary, superior, diagrammatic views, individual cushion elements used in elastic supporting and locating means for prosthetic menisci and the modes of their functional distortion;

FIG. 18 is a fragmentary, superior, diagrammatic view of a working embodiment of the elastic supporting and locating means of FIGS. 17a, b, c and d.

The figures are drawn to a variety of scales and no meaning or significance should be adduced from this fact.

With reference to FIGS. 3 and 10, the positioning and attachment of the horns of the lateral and medial menisci are depicted. With further reference to FIGS. 6¹ and 7² and particularly to FIG. 8³, it may be seen that, with progressive flexion of the knee joint, the menisci are progressively translated posteriorly. This is accompanied, to varying degrees, by posterolateral displacement in the case of the lateral meniscus and posteromedial displacement in the case of the medial meniscus. As the menisci are securely anchored by their horns, the result of such displacement is an elastic distortion of the menisci from their natural shapes and a stretching of the horns with a concomitant reduction in their heights. An inspection of FIGS. 1 and 2 will give a better understanding of factors affecting meniscal displacement or translation. The elastic distortion of the menisci appears to be such as to permit a very rapid recovery of the menisci to their relaxed positions during rapid and repeated flexion of the knee joint. It can be argued that this facility is a natural development almost wholly applicable to the young and physically active, and that a person of middle-age or older following a sedentary lifestyle has little need of it. For older persons, providing the menisci are constrained within a suitable range of translation with their positioning regulated by condylar movement, they will adequately perform their principal functions of providing shock absorption and enlarged bearing surface area for the joint. Limitation of meniscal translation to a range corresponding to a maximum knee joint flexion of 120° will accommodate most sedentary activities. The principal means employed to effect such translational constraint of the menisci are locating bands extending substantially around the tibial condyles and fixed to the tibia. With reference to FIG. 12, femur 1 terminates at its distal end in medial condyle 11 and lateral condyle 12. Patella 3 is depicted free of its ligamentary support and positioned above the knee joint as would be the case with considerable knee joint flexion. Fibula 10 is joined to tibia 2 at the superior tibiofibular joint and to the femur by the lateral collateral ligament 9. Femur 1 is joined to the tibia by medial collateral ligament 8. Lateral meniscus 7 and its anterior horn 13 are depicted in place. Locating band 4 is supported on one or more attachment lugs 5 fixed to the surfaces immediately inferior to the proximal edge of tibia 2 by suitable fastenings 6. Using a suitable routing cutter, bone is removed from the tibia to permit said attachment lugs to be inset and positioned more or less flush with the bone surface. Said attachment lugs are optionally located, in the case of the lateral meniscus, in the anterolateral lateral, lateral and posterolateral zones. In the case of the medial meniscus, said attachment lugs are optionally located in the anteromedial, medial and posteromedial zones. Access to said zones is readily gained via an incision of approximately 30 millimetres, by suitably positioning the knee joint, by parting and/or displacement of the surrounding tendinous and ligamentary tissue and by detachment and retraction of the synovial capsule. Said locating band is made from a suitable metal alloy material and is sufficiently stiff to sustain all normal loadings. Suitable materials for said locating bands are well known in the art and include passivated nitinol, titanium, austenitic stainless steels (which may have tantalum, niobium or titanium coatings), cobalt-chromium alloys, passivated beryllium, beryllium-aluminium alloys, nickel-chromium alloys, nickel-chromium-beryllium alloys, cobalt-chromium alloys, zirconia, zirconia-toughened alumina, metal-carbon fibre composite and the like. Said locating bands are shaped to conform accurately to the peripheral shaping of the proximal edge of the tibia as determined radiographically. Said locating bands are optionally provided with downwardly extending locating lugs which abut the surfaces immediately inferior to the proximal edge of the tibia. Suitable inwardly directed locating pegs are optionally provided in said lugs, said pegs being accommodated in bores suitably located in said tibial surfaces. The free ends of said locating bands are optionally provided with downwardly directed pegs which are accommodated in suitably located bores made in the proximal (upper) surfaces of the tibia, said pegs acting to positively locate said ends. Other means of stabilising the ends of said locating bands are also optionally employed. Said prosthetic menisci are moulded from a suitable biocompatible elastomer (the base material), in the preferred embodiment, the elastomer being DSM-PTG Carbosil® 20 90A biocompatible silicone polycarbonate urethane, manufactured by DSM Biomedical, of 6167 RA Geleen, The Netherlands. The principal mechanical properties of the material are:

| Density | 1.16 g/cc |
|---|---|
| Hardness, Shore A | 90 |
| Tensile Strength, Ultimate | 42.6 MPa |
| Tensile Strength, Yield | 6.4 MPa |
| [symbol] | [symbol] |
| Flexural Modulus | 0.0407 GPa |
| Flexural Strength, yield | 1.90 MPa |
| Tear Strength | 87.7 kN/m |
| Taber Abrasion, mg/1000 Cycles | 57.0 |
| Compression Set | 15.0% |

The material combines the biocompatibility and biostability of conventional silicone elastomers with the processability and toughness of thermoplastic urethane elastomers. The material is non-cytotoxic and non-haemolytic, has a low-energy silicone surface, has outstanding oxidative stability, is hydrophobic, has high tensile strength and is optically clear. PurSil™ silicone-polyetherurethane and CarboSil™ silicone-polycarbonateurethane are true thermoplastic copolymers containing silicone in the soft segment. These high-strength thermoplastic elastomers are prepared through a multi-step bulk synthesis where polydimethylsiloxane (PSX) is incorporated into the polymer soft segment with polytetramethyleneoxide (PTMO) (PurSil) or an aliphatic, hydroxyl-terminated polycarbonate (CarboSil). The hard segment consists of an aromatic diisocyanate, MDI, with a low molecular weight glycol chain extender. The copolymer chains are then terminated with silicone (or other) Surface-Modifying End Groups™. Aliphatic (AL) versions of these materials, with a hard segment synthesized from an aliphatic diisocyanate, are also available. PurSil and CarboSil can be melt fabricated by conventional extrusion, injection molding, or compression molding techniques. Rod, pellet, and tubing extruded from these materials displays an excellent surface finish and low gel content. In addition, these materials are heat-sealable, readily blended with fillers, and easily post-formed. In an alternative embodiment, said elastomer is Tecoflex® SG-93A thermoplastic polyurethane elastomer (polyether), manufactured by Lubrizol Advanced Materials, Inc., of Cleveland, Ohio, USA, which has a nominal Shore A hardness of 87. This material is formulated especially for solution moulding. In other alternative embodiments, elastomer materials similar in characteristics to the Carbosil and Tecoflex products and having a hardness in the Shore A range 60 to 95 are used with the present invention. In the preferred embodiment, prostheses are sized and shaped according to radiographically-derived images of the condyles, although some success has been demonstrated in the selection of allograft meniscal replacements simply in relation to such factors as sex and height of a subject. A mould is created for the required size and final shaping (or selected from an available range of moulds) of a specific prosthetic meniscus. With reference to FIGS. 13 and 15, in order to better accommodate forces applied to the prosthetic meniscus, in the preferred embodiment, prosthetic meniscus 14 is made in a plurality of more or less parallel layers of said base material bonded or fused at interfaces 25. Said layers of said base material are separated by thin sheets 16 of load-carrying material of suitable tensile strength. Said layers of said base material optionally vary in thickness according to the location within a prosthetic meniscus and number between 2 and 12. In a first embodiment, said load carrying material takes the form of a thin, flexible sheet material, such as Kevlar®. Said sheet material ranges in thickness from 0.005 to 0.1 millimetre. The thickness and extent of said sheet material optionally varies according to the location within a prosthetic meniscus. With specific reference to FIG. 15, in the preferred embodiment, a plurality of apertures 22 is provided in said sheet material to facilitate bonding or fusing of one layer of said base material to another, said apertures being of any suitable shape and of an arrangement such as to leave intact zones capable of satisfactorily carrying the radial and circumferential loads applied to said sheet material. In a second embodiment (not shown), said layers of said base material are separated by arrays of fibres of a material of suitable tensile strength, said fibres also being orientated to conform to the known radial and circumferential load paths. Photoelasticity methods are optionally employed to determine the direction and magnitude of stresses applied to a meniscus at various loadings. Said fibres are captured between said layers of said base material when they are bonded or fused together. In the preferred embodiment, said fibres are made from a polymer, such as Kevlar® or suitable carbon fibres. In an alternative embodiment (not shown), said fibres are distributed throughout a said prosthetic meniscus in a random way. With reference to FIG. 5, Young's modulus (or tensile modulus) values for locations within the human menisci in tension are shown (values in MPa). It will be noted that the values are well below those of most polymer materials. For example, Kevlar (aramid) has a tensile modulus normally in the range 83 to 186 GPa. With reference to FIG. 4, the disposition and arrangement of the natural fibrous reinforcement is depicted. In the preferred embodiment, said layers of said base material are assembled by thermal fusion or bonding with the final shaping occurring in a mould created for the purpose. Said final mould is finely finished to provide a glass-smooth finish to the upper and lower surfaces of the final prosthetic meniscus. In the preferred embodiment, thermal fusing of said layers of said base material one to another is effected by heating two surfaces to be joined above their fusion temperatures by contact with a hot plate and then firmly urging them together. Also in the preferred embodiment, bonding together of said layers of said base material is effected using one of the permanent biocompatible adhesives which are well known in the art.

In an alternative embodiment (not shown), either or both bearing surfaces of said menisci are provided with thin layers of a softer, more compliant base material, the thickness of said thin layers being preferably in the range 0.1 to 2.0 millimetres. By providing a more compliant bearing surface, this embodiment is better able to achieve microelastohydrodynamic lubrication. In another alternative embodiment (not shown), said menisci are made completely from a softer, more compliant base material. Menisci of this embodiment are employed temporarily during repair of femoral or tibial articular cartilage and are subsequently replaced with menisci made from a harder base material.

In alternative embodiments, said prosthetic menisci are made from one or more of the synthetic polypeptide materials of the type taught by Keeley et al in Patent No. WO 2008/140703 A2⁵. These materials comprise at least three consecutive beta-sheet/beta-turn structures and at least one crosslinking amino acid residue that participates in crosslinking, wherein the crosslinking residue is distinct from the beta-sheet/beta-turn structures, each polypeptide is between 150 and 500 amino acids in length and the material is a solid or liquid. In particular aspects, each beta-sheet structure may comprise from 3 to about 7 amino acid residues. In some embodiments, the amino acid sequences of the crosslinked polypeptides are the same; while in other embodiments the amino acid sequences of the crosslinked polypeptides are different. In some embodiments, the material further comprises a reinforcing material, such as animal material, a synthetic material or metal. In other embodiments, the material further comprises a non-protein hydrophilic polymer. In some embodiments, the material further comprises glycosaminoglycan moieties, such as hyaluronan moieties. In some embodiments, the material comprises a mixture of crosslinked polypeptides and glycosaminoglycan moieties. In other embodiments, the crosslinked polypeptides are covalently linked to the glycosaminoglycan moieties. In some embodiments, the material is solid and may be in the form of pads, sheets and ligament-like structures. In other embodiments, the material is a liquid, such as a solution or suspension.

With reference to FIGS. 14 and 16, locating band 4 is supported on one or more attachment lugs 5 fixed to the surfaces immediately inferior to the proximal edge of tibia 2 by suitable fastenings passing through apertures 21. In the preferred embodiment, said apertures are countersunk to permit the heads of said fastenings to be flush with the external surfaces of said lugs. Said attachment lugs are optionally located, in the case of the lateral meniscus, in the anterolateral lateral, lateral and posterolateral zones. In the case of the medial meniscus, said attachment lugs are optionally located in the anteromedial, medial and posteromedial zones. Although attachment lug 5 is depicted as orientated parallel to the inner surface of said locating band, in application, said lugs are joggled or angled, as required, to conform to the tibial surface. The outer surface 4 of said locating band is made curved and is finely finished. With additional reference to FIG. 11, the location of the inner surface 30 of said locating band is indicated by line 31. In a first embodiment of the present invention, prosthetic meniscus 14 is constrained within a suitable range of translation by bridles 19, the inner ends of which are embedded in said prosthetic meniscus and the outer ends of which are fixed to said locating band. It can be seen from the figure that, when said prosthetic meniscus is displaced such that its inner margin moves from position 27 to position 27a (depicted in broken line), said bridles are displaced from positions 19 to positions 19a (depicted in broken line). The lowermost bridle (as depicted in the figure) undergoes little displacement and is merely slightly slackened. In order to ensure that said prosthetic meniscus is reliably constrained within the desired range of translation, said bridles are provided in larger numbers around the periphery of said prosthetic meniscus between said meniscus and said locating band. In the preferred embodiment, said bridles locating a specific prosthetic meniscus number between 5 and 40. Also in the preferred embodiment, said bridles are made in looped form, the inner ends passing around an anchor element 18 in the form of a metal wire or monofilament of a stiffly elastic polymer material embedded in said prosthetic meniscus and the outer ends passing in and out through a pair of closely-spaced apertures (one shown numbered 23) in said locating band, the join (not shown) of said bridle ends being recessed in circumferential groove 24 cut in the exterior surface of said locating band. In the preferred embodiment, said bridles are a firm fit in apertures 23 and the inner openings of said apertures are flared to minimise the possibility of chafing damage to said bridles. In the preferred embodiment, said bridles are braided from a large number of fine Kevlar® fibres in the manner well known in the art and their outer ends are joined by suitable knots which are locked by impregnation with a suitable adhesive. In alternative embodiments, said bridles are spun or braided from a large number of fibres of any material having a suitable tensile strength.

With reference to FIG. 9, it can be seen that the native meniscus 34 is attached to synovial capsule 37 via ligamentary connection 38 and thence, via said synovial capsule, to tibial articular cartilage 36. Femoral articular cartilage 35 has freedom of movement in relation to said meniscus and said tibial articular cartilage. It will be seen that a space exists between the meniscus proper and the synovial membrane, this space, normally occupied by ligamentary connection 38, becoming available when said native meniscus and said ligamentary connection are removed. In a first alternative embodiment (not shown), the annular zone between the circumferential face of a said prosthetic meniscus and the inner face of said locating band is filled with a cushion element in the form of a closed-cell foam material formed from a suitable elastic polymer. The shaping and elastic character of said foam material permits ready translation of said prosthetic meniscus but continuously urges said meniscus towards its natural position. In the preferred embodiment, said foam material is made locally stiffer in some zones to provide a greater force to urge a said prosthetic meniscus towards its natural position. In this embodiment, said foam material is fixed to said prosthetic meniscus and to said locating band and has a cross-sectional shape which is square or rectangular. In a second alternative embodiment (not shown), the annular zone between the circumferential face of a said prosthetic meniscus and the inner face of said locating band is filled with a cushion element in the form of a tube pressurised to a suitable pressure with a suitable gas or partially filled with a suitable liquid or gel which permits ready translation of said prosthetic meniscus but continuously urges said meniscus towards its natural position. In the preferred embodiment, said tube is made locally thicker in some zones to provide a greater force to urge a said prosthetic meniscus towards its natural position. In this embodiment, said tube is fixed to said prosthetic meniscus and to said locating band and has a relaxed cross-sectional shape which is round, oval, square or rectangular.

With reference to FIG. 17a, in a third preferred embodiment, the annular zone between the circumferential face of a said prosthetic meniscus and the inner face of said locating band is filled with a plurality of cushion elements 39 made from a suitable elastomeric material and having a circular relaxed form. Said cushion elements are preferably moulded integrally with edge panels 40, 41 which are fixed to said circumferential face of a said prosthetic meniscus and to the inner face of said locating band. Said cushion elements are made with an inner diameter of between 1.0 and 10 millimetres, with a wall thickness of between 0.5 and 3 millimetres and a height to suit the edge thickness of said prosthetic meniscus. With reference to FIG. 17b, said cushion elements are able to be flattened to reduce the clearance between said prosthetic meniscus and said locating band. With reference to FIG. 17c, said cushion elements are able to be stretched out or extended to increase the clearance between said prosthetic meniscus and said locating band. With reference to FIG. 17d, said cushion elements are able to be rollingly distorted to permit independent longitudinal movement between attachment panels 40, 41 and, thereby, between said meniscus and said locating band.

With reference to FIG. 18, said cushion elements are preferably moulded in one or more rows, multiple rows being separated by separation panels 42 and complete arrays being edged by attachment panels 40, 41. In said arrays, said cushion elements are orientated with their axes parallel to the tibial axis and are preferably separated by sufficient distance to accommodate the flattened mode depicted in FIG. 17b without interference of adjacent elements. In the preferred embodiment, said arrays are made in more or less part-circular form to conform to the peripheral shaping of a said prosthetic meniscus. Also in the preferred embodiment, provision is made for a free flow of synovial fluid around said cushion elements, said separation panels and said attachment panels to prevent the development of hydraulic effects which might impede the translation of a said prosthetic meniscus. Said arrays permit ready translation of a said prosthetic meniscus but continuously urge said meniscus towards its natural position. In the preferred embodiment, said cushion elements are made locally thicker in some zones to provide a greater force to urge a said prosthetic meniscus towards its natural position.

In the preferred embodiment, anchor element 18 is made in the form of a wire of a stiffly elastic metal material, tapering towards each end. By extending substantially throughout the circumferential extent of a said prosthetic meniscus, said wire acts to elastically restore said meniscus to a natural shape after any distortion. Where said anchor element takes the form of a monofilament of stiffly elastic polymer material, a shaping band 17 of a stiffly elastic material is optionally fixed to the circumferential face of said prosthetic meniscus. In the preferred embodiment, said shaping band passes substantially around the circumferential extent of a said prosthetic meniscus and acts to elastically restore said meniscus to a natural shape after any distortion. In the preferred embodiment, said shaping band is made from a suitable elastic metal material, from a stiffly flexible engineering polymer material, from carbon fibre or any suitable composite. Where said shaping band is used with said bridles, suitable flared apertures 20 are provided in said shaping band. The ends of prosthetic menisci 14, 15 are optionally joined by straps 26 which transmit hoop stresses generated in said menisci. Transmission of said hoop stresses acts to maintain a more or less natural shaping of said prosthetic menisci during loadings tending to distort them. In the preferred embodiment, said straps are flat braided from fine Kevlar® fibres and their ends are securely embedded in said meniscus ends. In the preferred embodiment, said straps are coated with said base elastomer material, which is then treated in the manner described herein to make it attractive to dipalmitoylphosphatidylcholine, the most abundant phospholipid in synovial fluid. To more securely locate said locating bands, in the preferred embodiment, their ends 28, 29, 32, 33 are fixed to the proximal tibial surface. In this embodiment (not shown), said ends are provided with pegs which are accommodated within suitable bores made in the tibial surface or said ends are fixed to said tibial surface with suitable fastenings. Where said ends are fixed to the tibial surface, bone is removed using a suitable routing cutter to create short channels in which said ends are accommodated. In the preferred embodiment, said locating band ends are extended, turned through 90°, joggled and shaped to register with said channels.

Said moulds for the final shaping of said prosthetic menisci are made with polished surfaces to provide a glass-smooth finish to said menisci upper and lower bearing surfaces. To improve lubrication of said menisci by synovial fluid; said bearing surfaces are treated using a method⁴ which renders them attractive to dipalmitoylphosphatidylcholine (DPPC) by impregnating said surfaces with poly[2-methacryloyloxyethyl phosphorylcholine-co-n-butylmethacrylate] [poly(MPC-co-BMA)]. [poly (MPC-co-BMA)] is a biocompatible, lipid-attracting polymer soluble in solvent systems which also dissolve many polyurethanes. DPPC is the most abundant phospholipid in synovial fluid. In said method, the polyurethane elastomer is immersed in an ethanol solution containing BMA (0.3 mol l⁻¹) and benzoic peroxide (1 wt % to BMA) as a polymerization initiator for 15 hours, resulting in a slightly swollen surface. The material is lightly washed with ethanol and then immersed in an ethanol solution containing MPC (0.3 mol l⁻¹) for 30 minutes. After removal from the second solution, the material is blotted dry and then heated at 70° C. for 5 hours under an argon atmosphere to polymerize the monomers present in the surface of the material. Finally, the material is washed with ethanol and then dried en vacuo at room temperature for 24 hours. To improve the distribution of synovial fluid between the bearing surfaces of said prosthetic menisci and the femoral and tibial articular cartilage, a network of narrow channels is moulded into one or both said bearing surfaces. In the preferred embodiment, said channels have a width of between 0.25 and 2.0 millimetres, a depth of between 0.25 and 2.0 millimetres, have a part-spherical or other suitable cross-sectional shape, are separated by between 1.0 and 5.0 millimetres and are orientated more or less radially and circumferentially. Also for the same purpose, either or both said bearing surfaces are provided at some or all of the points of intersection of said channels with recesses orientated more or less normal to the surface at each point, having a depth of between 0.5 and 5.0 millimetres and a diameter of between 0.5 and 5.0 millimetres. For the same purpose, either or both said bearing surfaces are provided with recesses orientated more or less normal to the surface at each point, said recesses having a depth of between 0.5 and 5.0 millimetres, a diameter of between 0.5 and 5.0 millimetres and being separated from each other by a distance of between 0.5 and 10 millimetres. For the same purpose, said prosthetic menisci are provided with a plurality of ducts passing from said lower bearing surface to said upper bearing surface, said ducts being orientated more or less normal to said lower bearing surface, having a diameter of between 0.5 and 5.0 millimetres and being separated from each other by a distance of between 0.5 and 10 millimetres.

In the implantation of said prosthetic menisci, access is gained to the knee compartment via minimal incisions and separation or displacement of the tendinous and capsular tissue surrounding the joint. The native menisci are removed as required by surgically severing all of their tibial, ligamentary and capsular attachments with careful attention to haemostasis, a process well known in the art. Where only one said native meniscus is removed, the transverse geniculate ligament is severed at an appropriate length and sutured to the base of the anterior cruciate ligament. Said prosthetic menisci and said locating bands are selected for size and shape from radiographic images. Bone is removed as necessary to accommodate said attachment lugs and ends of said locating bands. Said prosthetic menisci are lubricated and correctly positioned between the femoral condyles, varus or vagus force being applied as necessary to open the joint. Fastenings are inserted to fix said attachment lugs and locating band ends to the tibia with said locating elements positioned between said locating bands and said prosthetic menisci. The synovial capsule is modified as required to fully enclose the joint, separated tissue is reinstated and the skin incisions are closed.

In an arthroscopic procedure, a said meniscus is made without anchor element 18 and shaping band 17 (both as depicted in FIG. 14) and with cushion elements of the type depicted in FIG. 18. Outer attachment panel (depicted as 40 in FIG. 18) is made with loops (not shown) to neatly accommodate an anchor element in the form of an elongated bar shaped, in its assembled form, to more or less conform to the shape of the proximal tibial edge. In the preferred embodiment, said bar is made from a suitable strong, stiff metal alloy material with a round or oval cross-sectional shape which tapers towards the ends. Said bar is made in two parts which are joined, as appropriate, at the lateral or medial position by joining means which are supported from the tibia by a suitable attachment lug. During the implantation process, the native meniscus is removed arthroscopically via a small incision in the manner well known in the art. A said prosthetic meniscus and associated cushion elements are folded into compact form and, using the same incision, extruded into the joint space via a suitable tubular guide and opened out into place. Said bar parts are then entered through said incision and inserted into said attachment panel loops. Said bar parts are joined and locked together at said joining means, bone is removed to create a suitable recess in the tibia and the attachment lug of said joining means is fixed to the tibia. In the preferred embodiment, said joining means take the form of a double-ended boss having shaped sockets which receive complementary shaped ends of said bar parts. Also in the preferred embodiment, bone is removed approximately at the points of insertion of the horns of the native meniscus to create recesses into which suitable sockets are fixed. When said bar parts are inserted, their ends are entered into said sockets to stabilise their positions. In another alternative embodiment (not shown), said cushion element takes the form of a tube which is empty during said process of implantation, said tube being subsequently pressurised to a suitable pressure with a suitable gas or partially filled with a suitable liquid or gel using suitable injection means.

Any feasible combination of the apparatus and/or method described herein should be taken to be disclosed by the specification.

### REFERENCES

1. Fu F. H., Thompson W. O. Motion of the Meniscus During Knee Flexion. In: Mow V. C., Arnoczky S. F., Jackson D. W., eds. Knee Meniscus: Basic and Clinical Foundations. New York: Raven Press, 1992: Chapter 6, Figs 20 and 21.
2. Vedi V., Williams A., Tennant S. J., Spouse E., Hunt D. M., Gedroyc W. M. W. (1998). Meniscal Movement: An In-vivo Study Using Dynamic MRI. The Journal of Bone & Joint Surgery (Br) 1999; 81-B37-41.
3. Hamamoto K., Tobimatsu M. D., Zabinska-Uroda K. (2004). Magnetic Resonance Imaging Evaluation of the Movement and Morphological Changes of the Menisci During Deep Knee Flexion. J. Phys. Ther. Sci. Vol. 16 No. 2, 2004
4. Williams III P. F., Powell G. L., Love B., Ishihara A., Nakabayashi N., Johnson R., LaBerge W. (1994). Fabrication and Characterization of Dipalmitoylphosphatidylcholine-attracting Elastomeric Material for Joint Replacements.
5. Keeley et al. International Patent No. WO 2008/140703 A2. Synthetic Peptide Materials for Joint Reconstruction, Repair and Cushioning.

1.-66. (canceled)

67. Prosthetic knee menisci to be implanted in place of deteriorated native menisci to prevent damage to the articular cartilage of the femoral and tibial condyles and to arrest the progressive development of osteoarthritis; said prosthetic menisci being sized and shaped to be congruent with the femoral and tibial condylar surfaces; being made from materials having elastomeric characteristics similar to those of native menisci; being made, effectively, in solid, unitary form by assembly from layers of solid material separated by layers of thin, flexible shed reinforcement material; having bearing surfaces treated chemically and/or physically to improve the efficiency of lubrication by synovial fluid; being restricted in translation within the interarticular space by a plurality of locating elements connecting said menisci to circumferentially-manged, more or less rigid, enclosing locating bands; having optional internal elements for stiffening and anchoring purposes, optional external elements for stiffening purposes and optional straps joining their ends to transmit hoop stresses.

68. The prosthetic knee menisci of claim 67 in which translation of a said meniscus within the interarticular space is reliably constrained by a plurality of circumferentially distributed locating elements situated more or less in the annular zone normally occupied by the ligamentary connection joining the native meniscus to the synovial membrane, said locating elements connecting said meniscus to and cooperating with circumferentially arranged, more or less rigid, enclosing locating bands supported above the proximal edges of the tibia on one or more attachment lugs fixed to the tibia; optionally in which said attachment lugs are inset into the tibia to be more or less flush with the tibial surface, said lugs being joggled or angled, as required, to permit said locating bands to conform to the peripheral shaping of the proximal edge of the tibia; optionally in which said attachment lugs are located, in the case of the lateral meniscus, in the anterolateral, lateral and posterolateral zones and, in the case of the medial meniscus, in the anteromedial, medial and posteromedial zones; optionally in which translation is limited to that corresponding to a maximum knee joint flexion of 120 degrees.

69. The prosthetic knee menisci of claim 67 in which said locating bands are made from a suitable stiff, metal alloy material, such as passivated nitinol, titanium, austenitic stainless steels (which may have tantalum, niobium or titanium coatings), cobalt-chromium alloys, passivated beryllium, beryllium-aluminium alloys, nickel-chromium alloys, nickel-chromium-beryllium alloys or cobalt-chromium alloys and is sufficiently strong to sustain normal loadings; optionally in which said locating bands are made from a non-metal material, such as zirconia, zirconia-toughened alumina or metal-carbon fibre composite; optionally in which said locating bands are shaped to confirm accurately to the radiographically-determined peripheral shaping of the proximal edge of the tibia the outer surface of said locating bands being finely finished; optionally in which said locating bands are provided with downwardly-extending lugs which act to locate said bands by abutting the surfaces immediately inferior to the proximal edge of the tibia, said lugs optionally having inwardly-directed locating pegs which engage complementary bores in the tibia; optionally in which the free ends of said locating bands are optionally provided with downwardly directed locating pegs which are accommodated in complementary bores in the proximal tibial surface; optionally in which other means are provided to stabilize the free ends of said locating bands.

70. The prosthetic knee menisci of claim 67 in which the base material from which they are manufactured is a suitable biocompatible and biostable elastomer having a hardness in the Shore A range 60 to 95; optionally in which said base material is silicone-polvetherurethane or silicone-polycarhonateurethane; optionally in which said base material is thermoplastic polyurethane elastomer (polyether); optionally in which copolymer chains of said base material are terminated with silicone or other surface-modifying end groups; optionally in which final sizing and shaping of a said meniscus is performed in a mould selected for the purpose, said mould being treated to provide a glass finish on the upper and lower surfaces of said meniscus; optionally in which the base material is assembled together in parallel layers by thermal fusing or by bonding with a biocompatible adhesive, said layers capturing between them load-carrying reinforcement material of suitable strength; optionally in which the thickness of said layers of base material varies according to the location within a said meniscus; optionally in which said layers of base material number between 2 and 12.

71. The prosthetic knee menisci of claim 67 in which said reinforcement material takes the form of aramid film in the thickness range 0.005 to 0.1 millimetre; optionally in which the thickness and extent of said reinforcement material varies according to the location within a said meniscus, optionally in which said reinforcement material is provided with a plurality of apertures to facilitate bonding or fusing of one layer of said base material to another, said apertures being of any suitable shape and of an arrangement such as to leave intact zones capable of satisfactorily carrying the radial and circumferential loads applied to said sheet material; optionally in which said layers of base material are separated by arrays of fibres of a material of suitable tensile strength, said fibres also being orientated to conform to the known radial and circumferential load paths and are captured between said layers of said base material when they are bonded or fused together; optionally in which said arrays of fibres are made from a polymer, such as aramid or suitable carbon fibres, optionally in which said fibres are distributed throughout the extent of a said prosthetic meniscus in a random way.

72. The prosthetic knee menisci of claim 67 in which photoelasticity methods are employed to determine the direction and magnitude of stresses applied to a meniscus at various loadings; optionally in which, to better achieve microelastohydrodynamic lubrication, either or both bearing surfaces of said menisci are provided with thin layers of a softer, more compliant base material, the thickness of said thin layers being in the range 0.1 to 2.0 millimetres.

73. The prosthetic knee menisci of claim 67 in which said menisci are made completely from a softer, more compliant base material, said menisci being employed temporarily during repair of femoral or tibial articular cartilage; optionally in which said menisci are made from synthetic polypeptide materials of the type taught by Keeley et al in Patent No. WO 2008/140703 A2, the materials comprising at least three consecutive beta-sheet/beta-turn structures and at least one crosslinking amino acid residue that participates in crosslinking, wherein the crosslinking residue is distinct from the beta-sheet/beta-turn structures and each polypeptide is between 150 and 500 amino acids in length; and/or in which each beta-sheet structure may comprise from 3 to about 7 amino acid residues; and/or in which the amino acid sequences of the crosslinked polypeptides are the same or different; and/or in which the material further comprises a reinforcing material, such as animal material, a synthetic material or metal; and/or in which the material further comprises a non-protein hydrophilic polymer; and/or in which the material further comprises glycosaminoglycan moieties, such as hyaluronan moieties; and/or in which the material comprises a mixture of crosslinked polypeptides and glycosaminoglycan moieties; and/or in which the crosslinked polypeptides are covalently linked to the glycosaminoglycan moieties; and/or in which the material is solid and may be in the form of pads, sheets and ligament-like structures.

74. The prosthetic knee menisci of claim 67 in which said locating elements take the form of a plurality of bridles having their inner ends embedded in said prosthetic meniscus and their outer ends fixed to said locating band; optionally in which the number of said bridles of a particular meniscus falls in the range 5 and 40; optionally in which said bridles are made in looped form, their inner ends passing around an anchor element in the form of a metal wire or monofilament of a stiffly elastic polymer material embedded within said prosthetic meniscus and their outer ends passing in and out through pairs of closely-spaced apertures provided in said locating band, joins of said bridle ends being recessed in circumferential groove provided in the exterior surface of said locating band; optionally in which said bridles are a firm fit in said apertures in said locating bands, the inner openings of said apertures being flared to minimise the possibility of chafing damage to said bridles; optionally in which said bridles are braided from a large number of fine aramid fibres in the manner well known in the art and their outer ends are joined by suitable knots which are locked by impregnation with a suitable adhesive; optionally in which said bridles are spun or braided from a large number of fine fibres of any material of suitable tensile strength; optionally in which said locating elements take the form of one or more cushion elements made from a closed-cell foam material formed from a suitable elastic polymer, the shaping and elastic character of said foam material permitting ready translation of a said prosthetic meniscus while continuously urging it towards its natural position; optionally in which said foam material is made locally stiffer in some parts of said annular zone to provide a greater force to urge a said prosthetic meniscus towards its natural position; optionally in which said polymer foam material is fixed to said prosthetic meniscus and/or to said locating band and has a square or rectangular cross-sectional shape.

75. The prosthetic knee menisci of claim 67 in which said locating elements take the form of one or more cushion elements in the form of a tube of suitably elastic material pressurised to a suitable pressure with a suitable gas or partially filled with a suitable liquid or gel which permits ready translation of a said prosthetic meniscus but continuously urges said meniscus towards its natural position; optionally in which said tube is made locally thicker in some parts of said annular zone to provide a greater force to urge a said prosthetic meniscus towards its natural position; optionally in which said tube is fixed to said prosthetic meniscus and/or to said locating band and has a relaxed cross-sectional shape which is circular, oval, square or rectangular; optionally in which said locating elements take the form of a plurality of cushion elements made from a suitable elastomeric material, said cushion elements having a tubular relaxed form and preferably being moulded integrally with edge panels fixed to the peripheral surface of a said prosthetic meniscus and/or to the inner face of said locating band, the axes of said elements being arranged more or less parallel to the tibial axis; said cushion elements being made with an inner diameter of between 1.0 and 10 millimetres, with a wall thickness of between 0.5 and 3 millimetres and a height more or less equal to the edge thickness of said prosthetic meniscus; said cushion elements being able to be flattened to reduce the clearance between said prosthetic meniscus and said locating band, to be stretched out or extended to increase the clearance between said prosthetic meniscus and said locating band, or to be rollingly distorted to permit independent longitudinal movement between said meniscus and said locating band.

76. The prosthetic knee menisci of claim 75 in which said cushion elements are preferably moulded in arrays of one or more rows, individual rows being separated by separation panels and complete arrays being edged by attachment panels fixed to the peripheral edge of said meniscus and/or to said locating band, said cushion elements being orientated with their tubular axes parallel to the tibial axis and preferably separated by sufficient distance to accommodate said flattening mode without interference of adjacent elements; optionally in which said arrays of cushion elements are made in more or less part-circular or arcuate form to conform to the peripheral shaping of a said prosthetic meniscus; optionally in which provision is made for a free flow of synovial fluid around said cushion elements, said separation panels and said attachment panels to prevent the development of hydraulic effects which might impede the translation of a said prosthetic meniscus; optionally in which said arrays of cushion elements permit ready translation of a said prosthetic meniscus but continuously urge said meniscus towards its natural position, cushion elements in some parts of said annular zone being made locally thicker to provide a locally greater restoring force.

77. The prosthetic knee menisci of claim 67 in which said internal elements are made in the form of wires of a stiffly elastic metal material, tapering towards each end, one said element extending substantially throughout the circumferential extent of a said prosthetic meniscus and acting to elastically restore said meniscus to a natural shape after any distortion; optionally in which said internal elements are made in the form of monofilaments of stiffly elastic polymer material, one said element extending substantially throughout the circumferential extent of a said prosthetic meniscus and optionally supplemented by an external element in the form of a shaping band of a stiffly elastic material fixed to the peripheral surface of said prosthetic meniscus, said shaping band passing substantially around the circumferential extent of said prosthetic meniscus and acting to elastically restore said meniscus to a natural shape after any distortion; optionally in which said shaping band is made from a suitable elastic metal material, from a stiffly flexible engineering polymer material, from carbon fibre or any suitable composite material, suitable flared apertures being provided in said shaping band where said bridles are required to pass through it.

78. The prosthetic knee menisci of claim 67 in which the ends of a said prosthetic meniscus are optionally joined by a strap which transmits hoop stresses generated in said meniscus, transmission of said hoop stresses acting to maintain a more or less natural shaping of said prosthetic menisci during loadings which might otherwise tend to distort them; optionally in which said straps are flat braided from fine aramid fibres and their ends securely embedded in said meniscus ends, said straps preferably being coated with said elastomer base material which is treated to make it attractive to lubricating constituents of synovial fluid.

79. The prosthetic knee menisci of claim 67 in which said locating bands are fixed to the proximal tibial surface, pegs on the ends of said locating bands being accommodated within suitable bores made in the tibial surface or the ends of said locating bands being fixed to said tibial surface with suitable fastenings; optionally in which, where the ends of said locating bands are fixed to the tibial surface, bone is removed using a suitable routing cutter to create short channels in which said ends are accommodated, said locating band ends preferably being extended, rotated as required through approximately 90°, joggled downwardly and shaped as required to register with said channels.

80. The prosthetic knee menisci of claim 67 in which, to improve lubrication of said menisci by synovial fluid, said bearing surfaces are treated to render them attractive to dipalmitoylphosphatidylcholine (DPPC) by impregnating said surfaces with poly[2-methacryloyloxyethyl phosphorylcholine-co-n-butylmethacrylate][poly(MPC-co-BMA)].

81. The prosthetic knee menisci of claim 80 in which a polyurethane elastomer is immersed in an ethanol solution containing BMA (0.3 mol l-1) and benzoic peroxide (1 wt % to BMA) as a polymerization initiator for 15 hours to produce a slightly swollen surface, the material then being lightly washed with ethanol and immersed in an ethanol solution containing MPC (0.3 mol l-1) for 30 minutes; after removal from the second solution, the material is blotted dry and then heated at 70° C. for 5 hours under an argon atmosphere to polymerize the monomers present in the surface of the material which is then washed with ethanol and then dried en vacuo at room temperature for 24 hours.

82. The prosthetic knee menisci of claim 67 in which, to improve the distribution of synovial fluid between the bearing surfaces of said prosthetic menisci and the femoral and tibial articular cartilage, a network of narrow channels is moulded into one or both said bearing surfaces, said channels preferably having a width of between 0.25 and 2.0 millimetres, having a depth of between 0.25 and 2.0 millimetres, having a part-spherical or other suitable cross-sectional shape, being separated by between 1.0 and 5.0 millimetres and being orientated more or less radially and circumferentially; optionally in which, to improve the distribution of synovial fluid, either or both said bearing surfaces are provided at some or all of the points of intersection of said channels with recesses orientated more or less normal to the surface at each point, said recesses having a depth of between 0.5 and 5.0 millimetres and a diameter of between 0.5 and 5.0 millimetres; optionally in which, to improve the distribution of synovial fluid, either or both said hearing surfaces are provided with recesses orientated more or less normal to the surface at each point, said recesses having a depth of between 0.5 and 5.0 millimetres and a diameter of between 0.5 and 5.0 millimetres, said recesses being separated from each other by a distance of between 0.5 and 10 millimetres; optionally in which, to improve the distribution of synovial fluid, said prosthetic menisci are provided with a plurality of ducts passing from said lower bearing surface to said upper bearing surface, said ducts being orientated more or less normal to said lower bearing surface, having a diameter of between 0.5 and 5.0 millimetres and being separated from each other by a distance of between 0.5 and 10 millimetres.

83. The prosthetic knee menisci of claim 76 in which said outer attachment panel of said locating elements of a prosthetic meniscus is made with loops to neatly accommodate a said internal element in the form of an elongated bar shaped, in its assembled form, to conform more or less to the shape of the proximal tibial edge, in the preferred embodiment said bar being made from a suitable strong, stiff metal alloy material with a round or oval cross-sectional shape which tapers towards the ends; said bar being made in two parts which are joined, following installation, at the lateral or medial position by joining means supported from the tibia by a suitable attachment lug; following positioning of said prosthetic meniscus in the interarticular space, said bar parts being inserted individually into said attachment panel loops and joined and locked together at said joining means; bone being removed to create a suitable recess in the tibia to accommodate said attachment lug; said joining means taking the form of a double-ended boss having shaped sockets which receive complementary shaped ends of said bar parts; optionally in which bone is removed from the tibia at the approximate points of insertion of the horns of the native meniscus to create recesses into which suitable sockets are permanently fixed, the ends of said internal elements being entered into said sockets to stabilise their positions.

84. The prosthetic knee menisci of claim 74 in which a single said cushion is employed, said element taking the form of a tube which is empty during implantation and subsequently pressurised to a suitable pressure with a suitable gas or partially filled with a suitable liquid or gel using suitable injection means.

85. A method of providing prosthetic knee menisci to be implanted in place of deteriorated native menisci to prevent damage to the articular cartilage of the femoral and tibial condyles and to arrest the progressive development of osteoarthritis; said method including the provision of prosthetic menisci being sized and shaped to be congruent with the femoral and tibial condylar surfaces; being made from materials having elastomeric characteristics similar to those of native menisci; being made, effectively, in solid, unitary form by assembly from layers of solid material separated by layers of thin, flexible sheet reinforcement material; having bearing surfaces treated chemically and/or physically to improve the efficiency of lubrication by synovial fluid; being restricted in translation within the interarticular space by a plurality of locating elements connecting said menisci to circumferentially-arranged, more or less rigid, locating bands; having optional internal elements for stiffening and anchoring purposes, optional external elements for stiffening purposes and optional straps joining their ends to transmit hoop stresses.

86. The method of providing prosthetic knee menisci of claim 85 in which, during implantation of said prosthetic menisci, access is gained to the knee compartment via minimal incisions and separation or displacement of the tendinous and capsular tissue surrounding the joint; the native menisci are removed as required by surgically severing all of their tibial, ligamentary and capsular attachments with careful attention to haemostasis; where only one said native meniscus is removed, the transverse geniculate ligament is severed at an appropriate length and sutured to the base of the anterior cruciate ligament; said prosthetic menisci and said locating bands are selected for size and shape from radiographic images and bone is removed as necessary to accommodate said attachment lugs and/or the ends of said locating bands and/or the ends of said internal elements; said prosthetic menisci are lubricated and correctly positioned between the femoral condyles, varus or vagus force being applied as necessary to open the joint; fastenings are inserted to fix said attachment lugs and locating band ends to the tibia with said locating elements positioned between said locating bands and said prosthetic menisci; the synovial capsule is modified as required to fully enclose the joint, separated tissue is reinstated and the skin incisions are closed; optionally in which a native meniscus is removed arthroscopically via a small incision and a said prosthetic meniscus and associated locating elements are folded into compact form, extruded into the joint space via a suitable tubular guide and opened out into place; said internal element parts are then entered through said incision and inserted into said prosthetic meniscus to engage said locating elements; said internal element parts are joined and locked together by joining means and bone is removed from the tibia to create a suitable recess to accommodate an attachment lug to fix said joining means to the tibia; said joining means taking the form of a double-ended boss having shaped sockets which receive complementary shaped ends of said internal element parts; bone being removed at the approximate points of insertion of the horns of the native meniscus to create recesses into which suitable sockets are permanently fixed, the ends of said internal element being entered into said sockets to stabilise their positions.
